(19) United States
(12) Patent Application Publication
(10) Pub. No.: US 2020/0138547 A1
(43) Pub. Date: May 7, 2020

(54) A MYOFUNCTIONAL ORTHODONTICS SYSTEM AND METHOD

(71) Applicant: Ahangari Enterprises Pty Ltd., Fortitude Valley (AU)

(72) Inventor: Emad AHANGARI, Fortitude Valley (AU)

(21) Appl. No.: 16/497,314

(22) PCT Filed: May 30, 2018
(86) PCT No.: PCT/AU2018/050526

§ 371 Date: Sep. 24, 2019

(30) Foreign Application Priority Data
May 30, 2017 (AU) 2017902038

(51) Int. Cl. A61C 7/08; A61B 5/00; H04Q 9/00; G08B 21/18; G16H 20/40; G16H 40/63

(52) U.S. Cl. CPC A61C 7/08; A61B 5/4833; A61B 5/682; H04Q 9/00; H04Q 2209/43; G16H 20/40; G16H 40/63; A61B 2562/0247; G08B 21/182

(57) ABSTRACT

An orthodontic system including one or more sensors configured to capture compliance data with reference to an orthodontic treatment and a controller coupled to the one or more sensors, the controller configured to control access to a media device according to the captured compliance data.

### TECHNICAL FIELD

The present invention relates to dentistry, and in particular to myofunctional orthodontics.

### BACKGROUND ART

Myofunctional orthodontics are used to help straighten teeth and jaws without using braces, particularly in children. In short, a myofunctional device (also known as an appliance, or mouthpiece) is worn by a user to address the poor oral habits that ultimately cause poor dental development, while also providing light forces to align the teeth.

Typically, a myofunctional device is prescribed for use one to two hours each day and while sleeping, together with a series of myofunctional exercises. The treatment generally requires the user to keep his or her mouth closed.

A problem with myofunctional devices, however, and with myofunctional orthodontics generally, is that compliance levels, particularly among children, are often low. If users do not wear the myofunctional device correctly, or for the prescribed time period, the myofunctional treatment is negatively impacted.

In the case of children, parents often supervise the children to ensure compliance with the myofunctional treatment. However, such supervision is time consuming and not particularly efficient. In particular, a child may remove the myofunctional device, or open their mouth, when their parents are no longer supervising the treatment.

As such, there is clearly a need for an improved myofunctional orthodontic system and method.

It will be clearly understood that, if a prior art publication is referred to herein, this reference does not constitute an admission that the publication forms part of the common general knowledge in the art in Australia or in any other country.

### SUMMARY OF INVENTION

The present invention is directed to orthodontic systems and methods, which may at least partially overcome at least one of the abovementioned disadvantages or provide the consumer with a useful or commercial choice.

With the foregoing in view, the present invention in one form, resides broadly in an orthodontic system including:

one or more sensors configured to capture compliance data with reference to an orthodontic treatment; and

a controller coupled to the one or more sensors, the controller configured to control access to a media device according to the captured compliance data.

Preferably, the controller is configured to determine compliance with the orthodontic treatment. Preferably, the controller is configured to limit access to one or more functions of the media device when compliance with the orthodontic treatment is not determined. Suitably, the controller is configured to prevent playback of media on the media device when compliance with the orthodontic treatment is not determined.

Preferably, the controller is configured to control access to the media device as it receives compliance data.

Preferably, the orthodontic treatment is performed at least partly with an orthodontic device. Preferably, the orthodontic treatment comprises a myofunctional orthodontic treatment with a myofunctional orthodontic device.

Preferably, the one or more sensors include a pressure sensor. Preferably, the pressure sensor is configured to capture compliance data in the form of a closed state of a mouth of the user. Preferably, the pressure sensor is configured to be received between upper and lower lips of the user.

Preferably, the system further includes a server, configured to receive and store the compliance data. Preferably, the server is configured to provide remote access to the compliance data by a health professional, such as an orthodontist.

Preferably, the controller is integral to the media device. Preferably, the controller is coupled to the one or more sensors by a wireless data interface.

Preferably, the system includes a compliance monitoring device comprising the one or more sensors.

Preferably, the compliance monitoring device includes a body. The body may include a processor and memory, configured to receive signal data from the sensors and provide compliance data to the controller according thereto.

The processor may be configured to filter the signal data to generate the compliance data. The processor may be configured to filter the signal data using one or more threshold values to determine whether the user's mouth is open or closed.

The processor may be configured to filter the sensor data over time, to remove outliers and irregularities in the sensor data.

The compliance monitoring device may include a battery, configured to power the compliance monitoring device.

Preferably, the body is configured to be worn by the user. The body may include one or more clips, configured to attach to an item of clothing of the user.

Preferably, the one or more sensors comprise a pressure sensor, coupled to the body by a flexible cord.

The compliance monitoring device may include an orthodontic device to be worn in the mouth of the user to provide the orthodontic treatment. The one or more sensors may be coupled to the orthodontic device. The one or more sensors may be incorporated into the orthodontic device.

Preferably, orthodontic device may be coupled to the body. The one or more sensors may be positioned intermediate to the orthodontic device and the body.

The body may be coupled to the orthodontic device. The body may be configured to be positioned outside of the user's mouth, and the orthodontic device inside the user's mouth.

Preferably, the system is configured to provide guidance to the user based upon the captured compliance data. The guidance may include instructions or tips.

The media of the media device may comprise video, audio, interactive media (e.g. games), Internet pages, or any other suitable media.

The system may be configured to indirectly control access to the media device. The system may be configured to interrupt a signal to the media device. The system may be configured to interrupt a power supply to the media device.

In another form, the invention resides broadly in a compliance monitoring device including:

one or more sensors configured to capture compliance data with reference to an orthodontic treatment; and

a data interface, coupled to the one or more sensors, the data interface configured to provide compliance data to a controller, wherein the controller is configured to control access to a media device according to the captured compliance data.

In another form, the invention resides broadly in a compliance monitoring method including:

capturing, using one or more sensors, compliance data with reference to an orthodontic treatment; and

controlling, using a controller coupled to the one or more sensors, access to a media device according to the captured compliance data.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 illustrates a myofunctional orthodontic system, according to an embodiment of the present invention;

FIG. 2 illustrates a compliance monitoring device of the system of FIG. 1, according to an embodiment of the present invention;

FIG. 3 illustrates a cross sectional view of a user using the system of FIG. 1;

FIG. 4 illustrates a compliance monitoring device of a myofunctional orthodontic system, according to an alternative embodiment of the present invention.

FIG. 5 illustrates a cross sectional view of a user using the compliance monitoring device of FIG. 4;

FIG. 6 illustrates a compliance monitoring device of a myofunctional orthodontic system, according to an alternative embodiment of the present invention.

FIG. 7 illustrates a schematic of a myofunctional orthodontic system, according to an embodiment of the present invention; and

FIG. 8 illustrates a myofunctional orthodontic compliance monitoring method, according to an alternative embodiment of the present invention.

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

### DESCRIPTION OF EMBODIMENTS

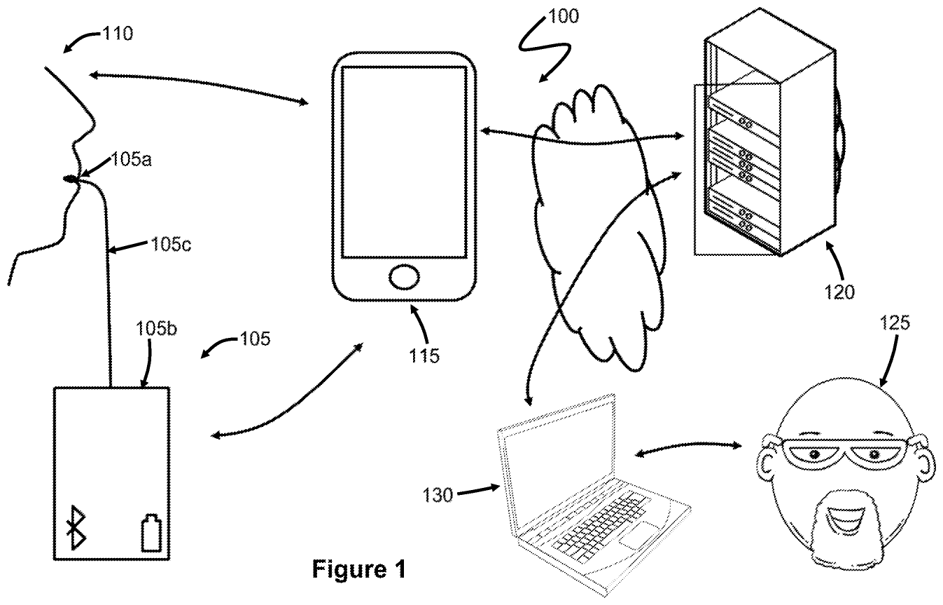

FIG. 1 illustrates a myofunctional orthodontic system 100, according to an embodiment of the present invention. The system 100 may be used to improve the rates of compliance of children undergoing myofunctional orthodontic treatment, which in turn results in a better orthodontic treatment.

The system 100 comprises a compliance monitoring device 105, which monitors compliance of a user 110 with reference to a myofunctional orthodontic treatment, and a multimedia device 115.

The multimedia device 115 is controlled to incentivise the user 110 in complying with the myofunctional orthodontic treatment. In particular, the compliance monitoring device 105 is coupled to a controller of the multimedia device 115, which is configured to control access to the device 115, or features thereof, based upon the monitored compliance of a user 110.

The controller may be implemented in software on the multimedia device 115, or as hardware coupled to the multimedia device. As an illustrative example, the controller may comprise an application which is configured to prevent playback of media when the user is not compliant with the treatment.

The multimedia device 115 is advantageously a smartphone or tablet computer with which the user 110 interacts to provide entertainment. As an illustrative example, the multimedia device may include (or provide access to) media such as video, audio, interactive media (e.g. games), Internet pages, or any other suitable media.

As illustrated in FIG. 3, the myofunctional orthodontic treatment comprises a myofunctional orthodontic device 305, which is placed in the mouth of the user 110, and helps straighten the teeth and jaws of the user. In particular, a myofunctional orthodontic device 305 is worn by a user to address the poor oral habits that cause poor dental development, such as mouth breathing, tongue thrusting and reverse swallowing, while also providing light forces to align the teeth into their natural position.

Compliance with the myofunctional orthodontic treatment requires the user 110 to keep his or her mouth closed during a treatment session, which may typically last between 1 and 2 hours. The compliance monitoring device 105 monitors such compliance using a pressure sensor 105a.

In particular, the pressure sensor 105a is placed between the lips 110a of the user 110, to detect whether the user's mouth is closed during a treatment session, and thus whether the user 110 is compliant with the myofunctional orthodontic treatment. In case the user is not compliant (e.g. his or her mouth is open), the playback of media on the media device 115 is stopped.

In place of the media, a screen is provided on the media device 115 that provides guidance to the user 110 based upon the captured compliance data. The guidance may include instructions (“Place sensor between lips and close mouth”) or tips, such as how to make the treatment more comfortable or efficient.

The guidance may be generated based upon the captured compliance data. For example, if the user 110 is repeatedly non-compliant, the guidance may become more basic illustrating the reason behind the treatment and why it is important. However, if the user 110 momentarily becomes non-compliant, the guidance may be a simple reminder that immediately disappears when the user becomes compliant again.

In the case of video, audio or interactive media (e.g. games), the video audio or interactive media stream may be paused, so that the user 110 is able to continue viewing or playing the media when the user becomes compliant again.

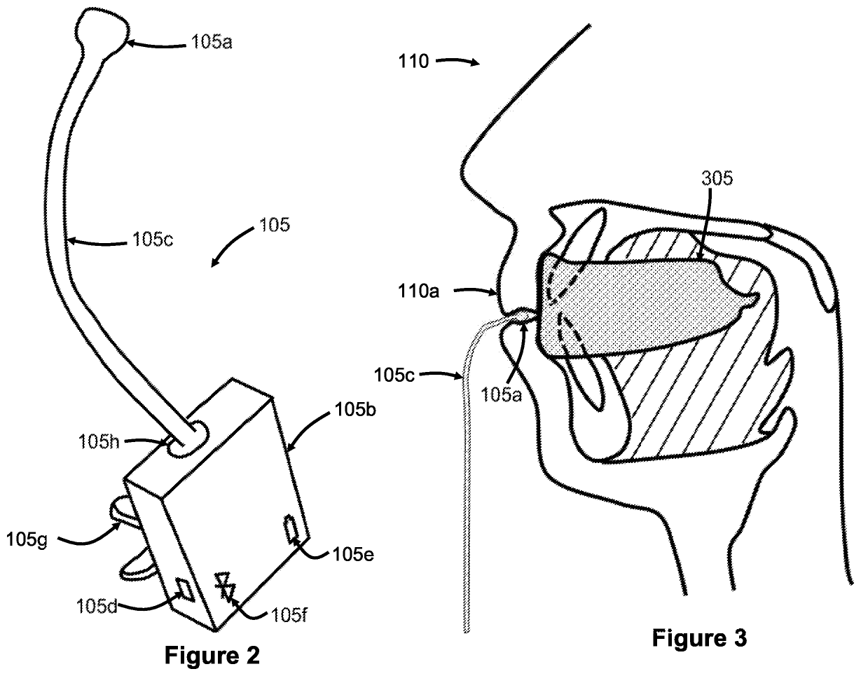

As best illustrated in FIG. 2, the compliance monitoring device 105 comprises a body 105b, which is coupled to the sensor 105a by a semi-rigid flexible cord 105c. The body 105b includes a flexible boot 105h, to enable the cord 105c to bend relative to the body 105b without applying pressure to the user's lips 110a.

The body 105b includes a processor and memory (not illustrated) and is configured to receive signal data from the sensor 105a and provide compliance data relating thereto to the controller of the media device 115 to enable control thereof. The compliance data is provided to the controller wirelessly, e.g. using Bluetooth.

The compliance data may comprise sensor data, which may be filtered, e.g. over time, to remove outliers and irregularities in the sensor data. In such case, the controller may analyse the sensor data to determine whether the user is compliant or not.

Alternatively, the sensor data may be analysed to generate compliance data in the form of whether the user is complaint or not. The processor may be configured to determine whether sensor data is above or below a threshold value to determine whether the user's mouth is open or closed (and thus compliant or not).

As such, analysis of the sensor data may be performed at the compliance monitoring device 105, at the controller of the media device 115, or a combination thereof.

The body 105b may include a rechargeable battery (not illustrated), configured to power the compliance monitoring device 105. In this regard, the body 105b further includes a charging port 105d, for charging the battery, and a battery status indicator 105e, for indicating a charge status of the battery. The body 105b further includes a wireless communication status indicator 105f, which can be useful in identifying a status of a wireless communications link with the controller, which is particularly useful in case of technical difficulties.

The body 105b is configured to be worn by the user 110 and includes clips 105g, configured to attach to an item of clothing of the user. As such, the pressure sensor 105a is supported by the user's clothing through the body 105b, and thus the device 104 is not supported by the user's lips 110a.

The pressure sensor 105a and the chord 105c are generally very small, so as to not obstruct the user unnecessarily as he or she is using the media device 115. As an illustrative example, the pressure sensor 105a may be about 5-8 mm in diameter, and the cord may be about 2 mm in diameter.

The media device 115 is further configured to send the compliance data to a server 120. The server 120 logs the data, and the data of a plurality of different users.

A parent or health professional 125, such as an orthodontist or dentist, may then log onto the server 120 using a computing device 130, to remotely access to the compliance data.

The server 120 may be configured to provide an overall schematic or rating of the user's performance, on a daily, weekly or monthly basis. As such, the health professional 125 may gauge short and long term motivation, and provide patient specific tips and advice based thereon. The tips and advice from the health professional 125 may be provided at non-compliance (as outlined above), or in general communication to the user.

As the health professional 125 is able to view the compliance data, he or she is not reliant on the user 110 evaluating his or her performance. In contrast, objective use and compliance data is provided to the health profession, which enables more accurate treatment to be provided.

According to certain embodiments, the compliance monitoring device 105 includes a unique identifier which is associated with the compliance data. When purchased, the compliance monitoring device is registered for the user 110, and details of the user, such as personal details, history, photos and the like are uploaded and associated with the compliance monitoring device 105.

According to certain embodiments, parents of the user 110 may log onto the server 120 and remotely configure a behaviour of the controller of the media device. For example, the parent may configure a sensitivity of the controller, a minimum treatment time, or any other parameter used by the controller. The server 120 is generally password in pin-code protected to prevent the user 110 from changing these settings.

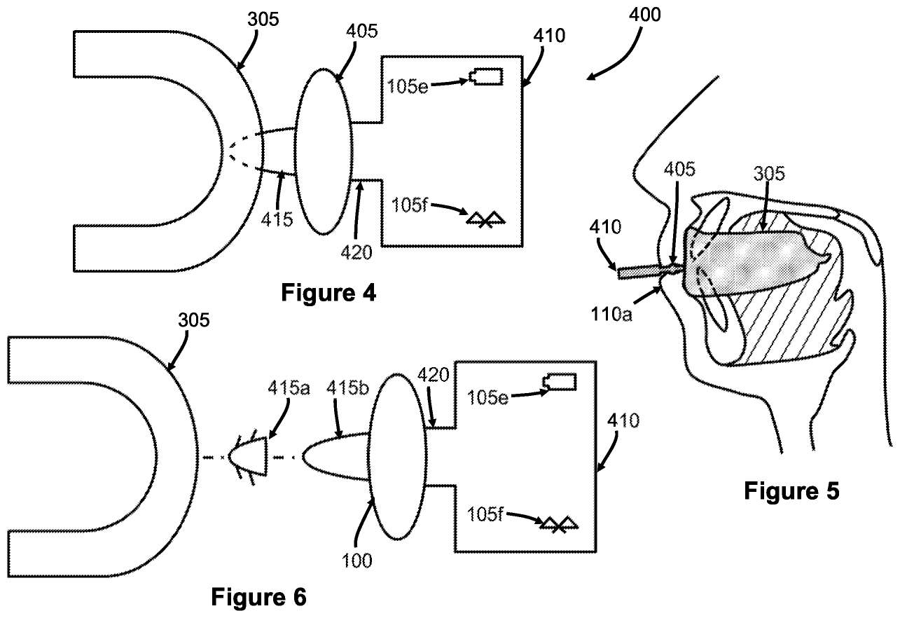

FIG. 4 illustrates a compliance monitoring device 400, according to an alternative embodiment of the present invention. The compliance monitoring device 400 is similar to the device 105, but is integrated with the orthodontic device 305.

The compliance monitoring device 400 includes a pressure sensor 405, similar to the pressure sensor 105a, and a body 410, similar to the body 105b. The sensor 105a is coupled to the orthodontic device 305 by an anchor 415, which extends into a front portion of the orthodontic device 305. The anchor 415 also functions as a spacer, to position the pressure sensor 405 between the lips 110a of the user 110 when the device 400 is in use.

The body 410 is similarly coupled to the pressure sensor 405 by a spacer 420, which is configured to place the body 410 away from the lips 110a. The anchor 415 and the spacer 420 extend outwardly from the orthodontic device 305 such that the body 410 is positioned immediately in front of the user's lips 110a.

In other words, the orthodontic device 305 is to be worn in the mouth of the user 110 and the body 410 is to be positioned outside of the user's mouth.

As illustrated in FIG. 6, the anchor 415 may comprise an insert portion 415a, for casting or insertion onto the orthodontic device 305, and a base portion 415b, which is configured to be received in the insert portion 415a. The insert portion 415a includes barbs, which prevent the insert portion from accidently dislodging from the orthodontic device 305.

In an alternative embodiment (not show), one or more sensors may be incorporated into the orthodontic device. Such sensors may monitor pressure of the device against the teeth, movement of the user's tongue, swallowing, or any other data.

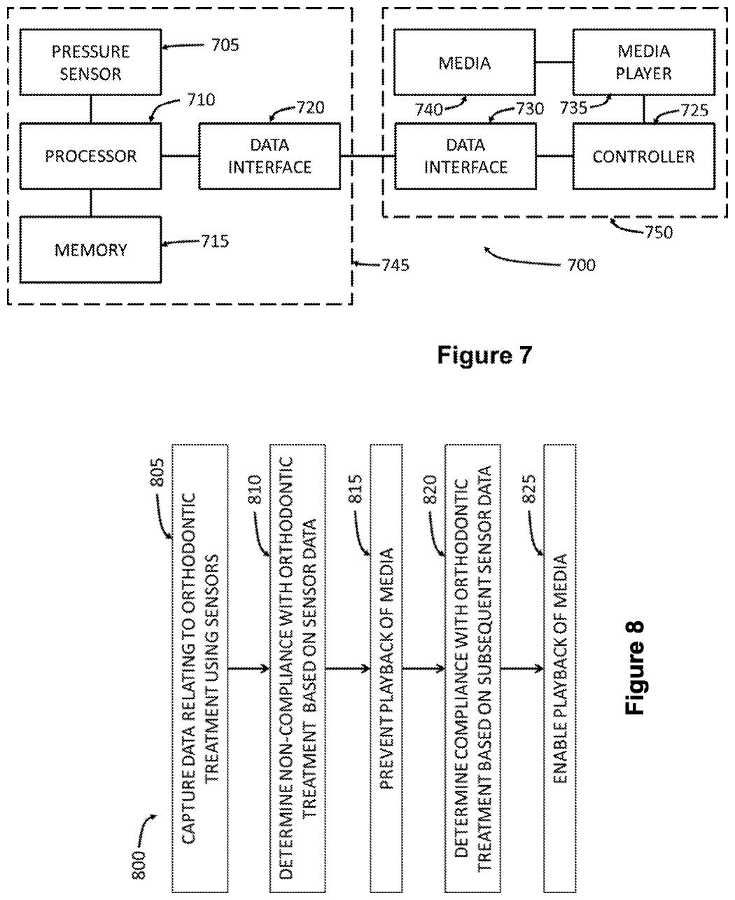

FIG. 7 illustrates a schematic of a myofunctional orthodontic system 700, according to an embodiment of the present invention. The system 700 may be similar or identical to the system 100 of FIG. 1.

The system 700 includes a pressure sensor 705, which is configured to sense a pressure between a user's lips. The sensor 705 is coupled to a processor 710, which in turn is coupled to a memory 715. The memory 715 includes instruction code executable by the processor 710 for receiving sensor data from the sensor 705 and determining whether the user is complaint based thereon.

When the user is compliant, a compliance signal is sent on a first data interface 720 to a controller 725, by a second data interface 730. The controller 725 is coupled to a media player 735, and is configured to enable playback of media 740 on the media player 735 only when the compliance signal is received.

When the user is non-compliant, a non-compliance signal is sent on the first data interface 720 to the controller 725. The controller 725 is configured to prevent playback of media 740 on the media player 735 only when the non-compliance signal is received.

The sensor 705, processor 710, memory 715 and first data interface 720 comprise a compliance monitoring device 745, and the controller 725, second data interface 730, media player 735 and media 740 comprise a controller device 750.

The skilled addressee will readily appreciate that in other embodiments, the system need not utilise both non-compliance and compliance signals. As an illustrative example, the system may be configured to enable playback of the media if and only if the compliance signal is present, and the lack of a compliance signal may be interpreted in a similar manner to the non-compliance signal mentioned above.

FIG. 8 illustrates a myofunctional orthodontic compliance monitoring method 800, according to an alternative embodiment of the present invention. The method 800 may, for example, be performed by the system 100 of FIG. 1.

At step 805, data relating to an orthodontic treatment is captured using one or more sensors. An example of a suitable sensor for capturing the data is a pressure sensor, as described above.

At step 810, non-compliance with an orthodontic treatment is determined based on the sensor data. The non-compliance may be determined based upon determining that the user's mouth is open, for example, as described above.

At step 815, playback of media on a media device is prevented. The media device may comprise a tablet computer, and the media may comprise a video or game, for example, as outlined above.

At step 820, compliance with the orthodontic treatment is determined based on the sensor data. The compliance may be determined based upon determining that the user's mouth is now shut again.

At step 825, playback of media on a media device is enabled again.

The playback of media on the media device is a reward to the user, and as such motivates the user to comply with the orthodontic treatment.

According to alternative embodiments (not illustrated), the compliance monitoring device need not connect directly with media device. In some embodiments an external device that disrupts the media device, such as a power or signal switch may be provided.

Advantageously, the systems and methods described above leverage children's attraction to the media devices (such as smart devices) to reward/incentivise them for complying with their orthodontic treatment. This may in turn cause more children to comply with their orthodontic treatment to be able to use their smart devices.

Furthermore, parents no longer have to strictly supervise their children's compliance with the orthodontic treatment, but instead are able to get independent and objective compliance data.

Finally, the treating medical practitioner is no longer reliant on a user's feedback regarding their compliance with the orthodontic treatment, as they are able to get objective compliance data. This also enables the medical practitioner to identify compliance issues early, and take appropriate corrective action.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. An orthodontic system including:
one or more sensors configured to capture compliance data with reference to an orthodontic treatment; and
a controller coupled to the one or more sensors, the controller configured to control access to a media device according to the captured compliance data.

2. The orthodontic system of claim 1, wherein the controller is configured to limit access to one or more functions of the media device when the captured compliance data indicates non-compliance with the orthodontic treatment.

3. The orthodontic system of claim 2, wherein the controller is configured to prevent playback of media on the media device the captured compliance data indicates non-compliance with the orthodontic treatment.

4. The orthodontic system of claim 1, wherein the orthodontic treatment is performed at least partly with an orthodontic device.

5. The orthodontic system of claim 4, wherein the orthodontic treatment comprises a myofunctional orthodontic treatment with a myofunctional orthodontic device.

6. (canceled)

7. The orthodontic system of claim 1, wherein the controller is:
integrated into the media device; and/or
coupled to the one or more sensors by a wireless data interface.

8. (canceled)

9. The orthodontic system of claim 1, including a compliance monitoring device including the one or more sensors, a processor and memory, configured to receive signal data from the one or more sensors, determine whether the signal data indicates compliance or non-compliance with the orthodontic treatment and provide compliance data to the controller according thereto.

10. The orthodontic system of claim 9, wherein the processor is configured to compare the signal data to one or more threshold values to determine compliance or non-compliance with the orthodontic treatment.

11. The orthodontic system of claim 10, wherein the processor is configured to filter the signal data over time, to remove outliers and irregularities in the signal data.

12. The orthodontic system of claim 9, wherein the compliance monitoring device includes a battery configured to power the compliance monitoring device.

13. The orthodontic system of claim 1, wherein the one or more sensors include at least one pressure sensor associated with an orthodontic device forming a part of the orthodontic treatment and configured to capture compliance data in the form of pressure data relating to a closed state of a mouth of the user.

14. The orthodontic system of claim 13, wherein the at least one pressure sensor is configured to be received between upper and lower lips of the user.

15. The orthodontic system of claim 1, wherein the compliance monitoring device includes a body configured to be worn externally by the user, the body including a processor and memory, configured to receive signal data from the one or more sensors, determine whether the signal data indicates compliance or non-compliance with the orthodontic treatment and provide compliance data to the controller according thereto.

16. The orthodontic system of claim 15, wherein:
the one or more sensors comprise a pressure sensor coupled to the body by a flexible extension; and/or
the compliance monitoring device includes an orthodontic device to be worn in the mouth of the user to provide the orthodontic treatment.

17. (canceled)

18. The orthodontic system of claim 13, wherein:
the one or more sensors are coupled to the orthodontic device; or
the one or more sensors are coupled to the orthodontic device and the one or more sensors are positioned intermediate to the orthodontic device and the body.

19. The orthodontic system of claim 13, wherein the one or more sensors are incorporated into the orthodontic device.

20. (canceled)

21. The orthodontic system of claim 1, wherein:
the system is configured to provide guidance to the user based upon the captured compliance data; and/or
upon non-compliance the controller is configured to interrupt a signal and/or power supply to the media device; and/or
the system further including a server, configured to receive and store the compliance data and to provide remote access to the compliance data by a health professional.

22. (canceled)

23. A compliance monitoring device including:
one or more sensors configured to capture compliance data with reference to an orthodontic treatment; and
a data interface, coupled to the one or more sensors, the data interface configured to provide compliance data to a controller, wherein the controller is configured to control access to a media device according to the captured compliance data.

24. A compliance monitoring method including:
capturing, using one or more sensors, compliance data with reference to an orthodontic treatment; and
controlling, using a controller coupled to the one or more sensors, access to a media device according to the captured compliance data.

25. An orthodontic system including:
one or more sensors configured to capture compliance data with reference to an orthodontic treatment; and
a controller coupled to the one or more sensors, the controller configured to control access to a media device according to the captured compliance data.
